(19) United States
(12) Patent Application Publication
(10) Pub. No.: US 2008/0243396 A1
(43) Pub. Date: Oct. 2, 2008

(54) Systems and methods for sub-genomic region specific comparative genome hybridization probe selection

(76) Inventors: Peter G. Webb, Menlo Park, CA (US); Jing Gao, San Jose, CA (US); B. Shane Giles, Fremont, CA (US); Sandra Tang, Saratoga, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/729,505

(22) Filed: Mar. 28, 2007

(51) Int. Cl. G06F 19/00; C12Q 1/68

(52) U.S. Cl. 702/20; 702/19; 427/2.14; 435/6

(57) ABSTRACT

Systems and methods for using the same to select one or more comparative genome hybridization (CGH) probes specific for a sub-genomic region of interest are provided. Also provided are computer program products for executing the subject methods.

### BACKGROUND

Comparative genomic hybridization (CGH) is one approach that has been employed to detect the presence and identify the location of amplified or deleted sequences in a genome. In one implementation of CGH, genomic DNA is isolated from normal reference cells, as well as from test cells. The two genomic DNAs are differentially labeled and then simultaneously hybridized to an array of surface-bound polynucleotide probes, e.g., an array of BACs, cDNAs or oligonucleotides. Chromosomal regions in the test cells which are at increased or decreased copy number can be identified by detecting regions where the ratio of signal from the two distinguishably labeled nucleic acids is altered. For example, those regions that have been decreased in copy number in the test cells will show relatively lower signal from the test nucleic acid than the reference compared to other regions of the genome. Regions that have been increased in copy number in the test cells will show relatively higher signal from the test nucleic acid.

### SUMMARY OF THE INVENTION

Aspects of the invention include systems for selecting a set of comparative genome hybridization (CGH) probes specific for a sub-genomic region that includes: (a) a communication module having an input manager for receiving input from a user and an output manager for communicating output to a user; (b) a database having stored thereon genomic information, at least one CGH probe group, and supporting information for each probe of the CGH probe group; and (c) a processing module having a genome region manager configured to identify a sub-genomic region of interest in response to at least one sub-genomic region identifier input by a user (where identification of the sub-genomic region is based in part on the genomic information in the database), and a probe selection manager configured to select a set of CGH probes specific for the sub-genomic region of intenrest. The probe selection manager selects a set of CGH probes based in part on the supporting information in the database. In certain embodiments, the set of CGH probes selected includes at least one probe from the CGH probe group(s) stored in the database.

In certain embodiments, the probe selection manager is further configured to select a set of CGH probes based on at least one probe-specific parameter input by a user.

In certain embodiments, the probe-specific parameter specifies one or more of: density of probes, types of probes, probe boundary, probe interval, minimum number of probes, maximum number of probes, probe computational score, gene confidence level, or any combination thereof.

In certain embodiments, the density of probes ranges from 1 probe/Mb to 10,000 probes/Mb, and as such may be 1 probe/Mb, 10 probe/Mb, 50 probes/Mb, 250 probes/Mb, 1000 probes/Mb, 5,000 probes/Mb, or 10,000 probes/Mb.

In certain embodiments, the types of probes specified is selected from one or more of: intron specific, exon specific, intergenic, intragenic, or a combination thereof.

In certain embodiments, the CGH probe group in the database includes one or more of: previously selected CGH probe group(s), a private CGH probe group(s), public CGH probe group(s), proprietary CGH probe group(s), curated CGH probe group(s), or any combination thereof.

In certain embodiments, the supporting information includes one or more of: probe length, computational score, probe annotation, or any combination thereof.

In certain embodiments, the genomic information includes one or more of: chromosomal information, polymorphism information, mutation information, transcriptome information, transcript mapping information, species information, or any combination thereof.

In certain embodiments, the sub-genomic region identifier includes information that includes one or more of: cytogenetic parameter, genomic sequence, gene identifier, chromosomal location, transcript identifier, species, chromosomal boundary and any combination thereof.

In certain embodiments, the probe selection manager is further configured to select a set of CGH probes based in part on at least one experimental parameter input by said user.

In certain embodiments, the experimental parameter includes one or more of: target sample preparation, assay format, assay parameter, and combinations thereof.

In certain embodiments, the processing module further includes a probe design manager configured to design at least one probe to include in the set of CGH probes, e.g., either automatically or when prompted by a user.

In certain embodiments, the system further includes a user domain configured to store CGH probe sets, e.g., either automatically or when prompted by a user.

In certain embodiments, the system further includes a probe fabrication module configured to fabricate a set of CGH probes, e.g., when prompted by a user.

In certain embodiments, the processing module further includes an array layout manager configured to design an array layout for a set of CGH probes, e.g., when prompted by a user.

In certain embodiments, the array layout manager is further configured to include in an array layout one or more of: replicate probes, normalization control probes, negative control probes, positive control probes, CGH probes specific for regions outside of the sub-genomic region of interest, or any combination thereof.

In certain embodiments, the system further includes an array fabrication module configured to fabricate an array based on an array layout, e.g., when prompted by a user.

In certain embodiments, the system further comprises a graphical user interface (GUI) linked to the communication module configured to prompt a user for input and to display output of the system to the user.

Aspects of the invention include methods of receiving a set of CGH probes specific for a sub-genomic region of interest by inputting an identifier for a sub-genomic region into a system of the invention and receiving one or more sets of CGH probes specific for the sub-genomic region.

Aspects of the invention include methods of selecting a set of CGH probes specific for a sub-genomic region of interest including the steps of: (a) providing a database having stored thereon genomic information, at least one CGH probe group, and supporting information for each probe of the CGH probe group(s); (b) identifying a sub-genomic region of interest based in part on the genomic information stored in the database; and (c) selecting a set of CGH probes specific for the sub-genomic region based in part on the supporting information stored in the database. In certain embodiments, the set of CGH probes includes at least one probe from a CGH probe group in the database.

In certain embodiments, the identifying step includes providing at least one sub-genomic region identifier that includes one of more of: cytogenetic parameter, genomic sequence, gene identifier, chromosomal location, transcript identifier, organism, chromosomal boundary, and combinations thereof; and identifying the sub-genomic region based in part on the sub-genomic region identifier.

In certain embodiments, the selecting step further includes specifying at least one probe-specific parameter; and selecting a set of CGH probes based in part on the probe specific parameter.

In certain embodiments, the at least one probe-specific parameter includes one or more of: density of probes, types of probes, probe boundary, minimum number of probes, maximum number of probes, probe computational score, gene confidence level, or any combination thereof.

In certain embodiments, the density of probes is selected from: 1 probe/Mb, 10 probe/Mb, 50 probes/Mb, 250 probes/Mb, 1000 probes/Mb, 5,000 probes/Mb, and 10,000 probes/Mb.

In certain embodiments, the probe type includes one or more of: intron specific, exonic specific, intergenic, intragenic, gene confidence level, or combinations thereof.

In certain embodiments, the CGH probe group(s) in the database includes one or more of: previously selected CGH probe group, private CGH probe group, public CGH probe group, proprietary CGH probe group, curated CGH probe group, or any combination thereof.

In certain embodiments, the supporting information is selected from one or more of: probe length, computational score, probe annotation, or any combination thereof.

In certain embodiments, the genomic information includes one or more of: chromosomal information, polymorphism information, mutation information, transcriptome information, species information, or any combination thereof.

In certain embodiments, the obtaining step further includes designing one or more probe in the set of CGH probes using a probe design algorithm.

In certain embodiments, the selecting step further includes submitting a set of CGH probes to a pairwise reduction algorithm.

In certain embodiments, the selecting step is further based on at least one experimental parameter.

In certain embodiments, the experimental parameter includes one or more of: target sample preparation, assay format, assay parameter, or any combination thereof.

In certain embodiments, the method further comprises storing a set of CGH probes in a database as one of the CGH probe groups.

In certain embodiments, the sub-genomic region identifier is provided using a graphical user interface (GUI).

In certain embodiments, the probe specific parameter is specified using a GUI.

In certain embodiments, the set of CGH probes is displayed on a GUI.

Aspects of the invention include methods of fabricating an array that includes: (a) selecting a set of CGH probes specific for a sub-genomic region of interest according to the methods of the invention (summarized above), (b) designing an array layout including the selected set of CGH probes; and (c) fabricating an array based on the array layout.

In certain embodiments, the array layout further includes one or more of: replicate probes, normalization control probes, negative control probes, positive control probes, CGH probes specific for regions outside of said sub-genomic region of interest, or any combination thereof.

Aspects of the invention include computer program products that include a computer readable storage medium having a computer program stored thereon, where the computer program, when loaded onto a computer, operates the computer to select a set of CGH probes specific for a sub-genomic region of interest identified by a user (e.g., based in part on one or more sub-genomic region identifiers specified by the user).

### BRIEF DESCRIPTIONS OF THE DRAWINGS

FIG. 1 illustrates a substrate carrying multiple arrays, such as may be fabricated by methods of the present invention.

FIG. 2 is an enlarged view of a portion of FIG. 1 showing multiple ideal spots or features.

FIG. 3 is an enlarged illustration of a portion of the substrate in FIG. 2.

FIG. 4 schematically illustrates an exemplary system of the present invention.

FIG. 5 provides exemplary graphical user interfaces that can be employed in the systems and methods of the present invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

By “array layout” is meant a collection of information, e.g., in the form of a file, which represents the location of probes that have been assigned to specific features of one or more array formats, e.g., a single array format or two or more array formats of an array set.

The phrase “array format” refers to a format that defines an array by feature number, feature size, Cartesian coordinates of each feature, and distance that exists between features within a given single array.

The phrase “array content information” is used to refer to any type of information/data that describes an array. Representative types of array content information include, but are not limited to: “probe-level information” and “array-level information”. By “probe-level information” is meant any information relating to the biochemical properties or descriptive characteristics of a probe. Examples include, but are not limited to: probe sequence, melting temperature (Tₘ), target gene or genes (e.g., gene name, accession number, etc.), location identifier information, information regarding cell(s) or tissue(s) in which a probe sequence is expressed and/or levels of expression, information concerning physiological responses of a cell or tissue in which the sequence is expressed (e.g., whether the cell or tissue is from a patient with a disease), chromosomal location information, copy number information, information relating to similar sequences (e.g., homologous, paralogous or orthologous sequences), frequency of the sequence in a population, information relating to polymorphic variants of the probe sequence (e.g., such as SNPs), information relating to splice variants (e.g., tissues, individuals in which such variants are expressed), demographic information relating to individual(s) in which the sequence is found, and/or other annotation information. By “array-level information” is meant information relating to the physical properties or intended use of an array. Examples include, but are not limited to: types of genes to be studied using the array, such as genes from a specific species (e.g., mouse, human), genes associated with specific tissues (e.g., liver, brain, cardiac), genes associated with specific physiological functions, (e.g., apoptosis, stress response), genes associated with disease states (e.g., cancer, cardiovascular disease), array format information, e.g., feature number, feature size, Cartesian coordinates of each feature, and distance that exists between features within a given array, etc.

A “data element” represents a property of a probe sequence, which can include the base composition of the probe sequence. Data elements can also include representations of other properties of probe sequences, such as expression levels in one or more tissues, interactions between a sequence (and/or its encoded products), and other molecules, a representation of copy number, a representation of the relationship between its activity (or lack thereof) in a cellular pathway (e.g., a signaling pathway) and a physiological response, sequence similarity to other probe sequences, a representation of its function, a representation of its modified, processed, and/or variant forms, a representation of splice variants, the locations of introns and exons, functional domains etc. A data element can be represented for example, by an alphanumeric string (e.g., representing bases), by a number, by “plus” and “minus” symbols or other symbols, by a color hue, by a word, or by another form (descriptive or nondescriptive) suitable for computation, analysis and/or processing for example, by a computer or other machine or system capable of data integration and analysis.

As used herein, the term “data structure” is intended to mean an organization of information, such as a physical or logical relationship among data elements, designed to support specific data manipulation functions, such as an algorithm. The term can include, for example, a list or other collection type of data elements that can be added, subtracted, combined or otherwise manipulated. Exemplary types of data structures include a list, linked-list, doubly linked-list, indexed list, table, matrix, queue, stack, heap, dictionary, flat file databases, relational databases, local databases, distributed databases, thin client databases and tree. The term also can include organizational structures of information that relate or correlate, for example, data elements from a plurality of data structures or other forms of data management structures. A specific example of information organized by a data structure of the invention is the association of a plurality of data elements relating to a gene, e.g., its sequence, expression level in one or more tissues, copy number, activity states (e.g., active or non-active in one or more tissues), its modified, processed and/or variant forms, splice variants encoded by the gene, the locations of introns and exons, functional domains, interactions with other molecules, function, sequence similarity to other probe sequences, etc. A data structure can be a recorded form of information (such as a list) or can contain additional information (e.g., annotations) regarding the information contained therein. A data structure can include pointers or links to resources external to the data structure (e.g., such as external databases). In one aspect, a data structure is embodied in a tangible form, e.g. is stored or represented in a tangible medium (such as a computer readable medium).

The term “object” refers to a unique concrete instance of an abstract data type, a class (that is, a conceptual structure including both data and the methods to access it) whose identity is separate from that of other objects, although it can “communicate” with them via messages. In some occasions, some objects can be conceived of as a subprogram which can communicate with others by receiving or giving instructions based on its, or the others' data or methods. Data can consist of numbers, literal strings, variables, references, etc. In addition to data, an object can include methods for manipulating data. In certain instances, an object may be viewed as a region of storage. In the present invention, an object typically includes a plurality of data elements and methods for manipulating such data elements.

A “relation” or “relationship” is an interaction between multiple data elements and/or data structures and/or objects. A list of properties may be attached to a relation. Such properties may include name, type, location, etc. A relation may be expressed as a link in a network diagram. Each data element may play a specific “role” in a relation.

As used herein, an “annotation” is a comment, explanation, note, link, or metadata about a data element, data structure or object, or a collection thereof. Annotations may include pointers to external objects or external data. An annotation may optionally include information about an author who created or modified the annotation, as well as information about when that creation or modification occurred. In one embodiment, a memory comprising a plurality of data structures organized by annotation category provides a database through which information from multiple databases, public or private, may be accessed, assembled, and processed. Annotation tools include, but are not limited to, software such as BioFerret (available from Agilent Technologies, Inc., Palo Alto, Calif.), which is described in detail in application Ser. No. 10/033,823 filed Dec. 19, 2001 and titled “Domain-Specific Knowledge-Based Metasearch System and Methods of Using.” Such tools may be used to generate a list of associations between genes from scientific literature and patent publications.

As used herein an “annotation category” is a human readable string to annotate the logical type that an object, comprising its plurality of data elements, represents. Data structures that contain the same types and instances of data elements may be assigned identical annotations, while data structures that contain different types and instances of data elements may be assigned different annotations.

As used herein, a “probe sequence identifier” or an “identifier corresponding to a probe sequence” refers to a string of one or more characters (e.g., alphanumeric characters), symbols, images or other graphical representation(s) associated with a probe sequence comprising a probe sequence such that the identifier provides a “shorthand” designation for the sequence. In one aspect, an identifier comprises an accession number or a clone number. An identifier may comprise descriptive information. For example, an identifier may include a reference citation or a portion thereof.

The phrase “best-fit” refers to a resource allocation scheme that determines the best result in response to input data. The definition of ‘best’ may vary depending on a given set of predetermined parameters, such as sequence identity limits, signal intensity limits, cross-hybridization limits, Tₘ, base composition limits, probe length limits, distribution of bases along the length of the probe, distribution of nucleation points along the length of the probe (e.g., regions of the probe likely to participate in hybridization, secondary structure parameters, etc. In one aspect, the system considers predefined thresholds. In another aspect, the system rank-orders fit. In a further aspect, the user defines his or her own thresholds, which may or may not include system-defined thresholds.

The terms “system” and “computer-based system” refer to the hardware means, software means, and data storage means used to analyze the information of the present invention. The minimum hardware of the computer-based systems of the present invention comprises a central processing unit (CPU), input means, output means, and data storage means. As such, any convenient computer-based system may be employed in the present invention. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, UBS, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any convenient method. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

In certain embodiments, a system includes hardware components which take the form of one or more platforms, e.g., in the form of servers, such that any functional elements of the system, i.e., those elements of the system that carry out specific tasks (such as managing input and output of information, processing information, etc.) of the system may be carried out by the execution of software applications on and across the one or more computer platforms represented of the system. The one or more platforms present in the subject systems may be any convenient type of computer platform, e.g., such as a server, main-frame computer, a work station, etc. Where more than one platform is present, the platforms may be connected via any convenient type of connection, e.g., cabling or other communication system including wireless systems, either networked or otherwise. Where more than one platform is present, the platforms may be co-located or they may be physically separated. Various operating systems may be employed on any of the computer platforms, where representative operating systems include Windows, MacOS, Sun Solaris, Linux, OS/400, Compaq Tru64 Unix, SGI IRIX, Siemens Reliant Unix, and others. The functional elements of system may also be implemented in accordance with a variety of software facilitators, platforms, or other convenient method.

Items of data are “linked” to one another in a memory when the same data input (for example, filename or directory name or search term) retrieves the linked items (in a same file or not) or an input of one or more of the linked items retrieves one or more of the others.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In general, synthesis of nucleic acids of this type utilizes an initial substrate-bound monomer that is used as a building-block in a multi-step synthesis procedure to form a complete nucleic acid. A “biomonomer” references a single unit, which can be linked with the same or other biomonomers to form a biopolymer (e.g., a single amino acid or nucleotide with two linking groups, one or both of which may have removable protecting groups).

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise 2-50 monomers, including 2-20, and including 3-10 monomers.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

A “biopolymer” is a polymer of one or more types of repeating units. Biopolymers are typically found in biological systems (although they may be made synthetically) and may include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” may include DNA (including cDNA), RNA, oligonucleotides, and PNA and other polynucleotides as described in U.S. Pat. No. 5,948,902 and references cited therein (all of which are incorporated herein by reference), regardless of the source.

The term “biomolecular probe” or “probe” means any organic or biochemical molecule, group or species of interest having a particular sequence or structure. In certain embodiments, a biomolecular probe may be formed in an array on a substrate surface. Exemplary biomolecular probes include polypeptides, proteins, oligonucleotide and polynucleotides.

The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the substrate.

The term “sample” as used herein relates to a material or mixture of materials, typically, although not necessarily, in fluid form, containing one or more components of interest.

A biomonomer fluid or biopolymer fluid refers to a liquid containing either a biomonomer or biopolymer, respectively (typically in solution).

The term “peptide” as used herein refers to any polymer compound produced by amide formation between an α-carboxyl group of one amino acid and an α-amino group of another group.

The term “oligopeptide” as used herein refers to peptides with fewer than 10 to 20 residues, i.e., amino acid monomeric units.

The term “polypeptide” as used herein refers to peptides with more than 10 to 20 residues.

The term “protein” as used herein refers to polypeptides of specific sequence of more than 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from 10 up to 200 nucleotides in length, e.g., from 25 to 200 nt, including from 50 to 175 nt, e.g. 150 nt in length

The term “polynucleotide” as used herein refers to single- or double-stranded polymers composed of nucleotide monomers of generally greater than 100 nucleotides in length.

An “array,” or “chemical array” used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. As such, an addressable array includes any one or two or even three-dimensional arrangement of discrete regions (or “features”) bearing particular biopolymer moieties (for example, different polynucleotide sequences) associated with that region and positioned at particular predetermined locations on the substrate (each such location being an “address”). These regions may or may not be separated by intervening spaces. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either precursor units (such as nucleotide or amino acid monomers) in the case of in situ fabrication, or the previously obtained biomolecule, e.g., polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein.

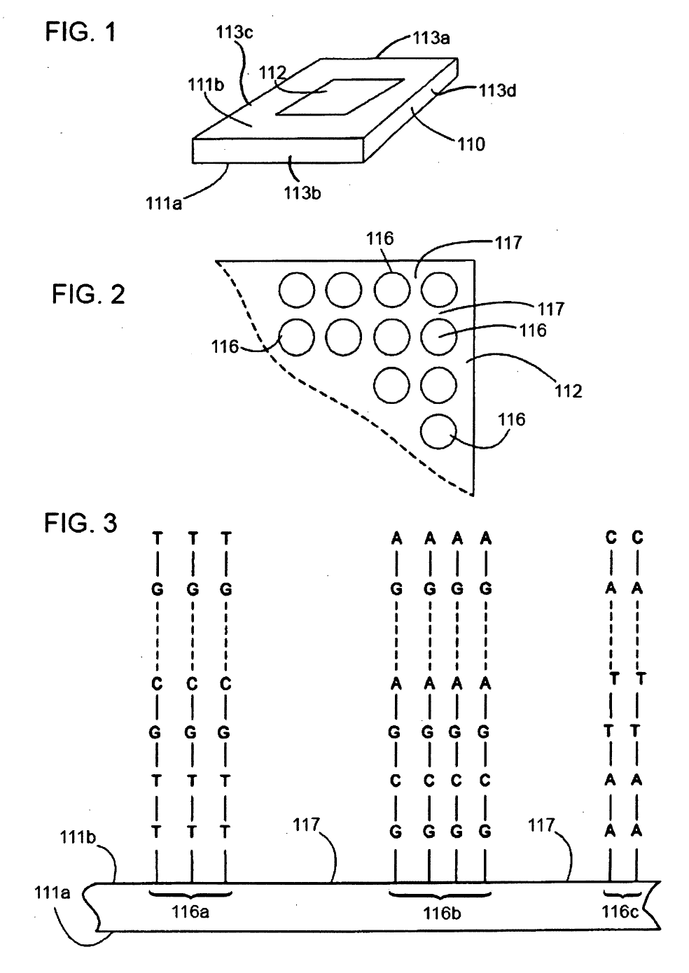

An exemplary chemical array is shown in FIGS. 1-3, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the surface 111b, with regions of the rear surface 111b adjacent the opposed sides 113c, 113d and leading end 113a and trailing end 113b of slide 110, not being covered by any array 112. A second surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymer ligands, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) between the rear surface 111b and the first nucleotide. Any convenient linker may be used.

Substrate 110 may carry on surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

The substrate may be porous or non-porous. The substrate may have a planar or non-planar surface.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

The term “substrate” as used herein refers to a surface upon which marker molecules or probes, e.g., an array, may be adhered. Glass slides are the most common substrate for biochips, although fused silica, silicon, plastic and other materials are also suitable.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

“Hybridizing” and “binding”, with respect to polynucleotides, are used interchangeably.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of 0.02 molar at pH 7 and a temperature of at least 50° C. or 55° C. to 60° C.; or, a salt concentration of 0.15 M NaCl at 72° C. for 15 minutes; or, a salt concentration of 0.2×SSC at a temperature of at least 50° C. or 55° C. to 60° C. for 15 to 20 minutes; or, the hybridization complex is washed twice with a solution with a salt concentration of 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than 5-fold more, typically less than 3-fold more. Other stringent hybridization conditions may also be employed, as appropriate.

“Contacting” means to bring or put together. As such, a first item is contacted with a second item when the two items are brought or put together, e.g., by touching them to each other.

“Depositing” means to position, place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

### DETAILED DESCRIPTION

Systems and methods for selecting a set of comparative genome hybridization (CGH) probes specific for a sub-genomic region of interest are provided.

Aspects of the invention include systems configured to select a set of CGH probes for a sub-genomic region based on at least one sub-genomic region identifier, e.g., an identifier that has been input by a user. The subject systems include a communications module and a processing module, where the processing module includes a genome region manager configured identify a sub-genomic region of interest based in part on a sub-genomic region identifier and a probe selection manager configured to select a set of CGH probes specific for the sub-genomic region identified by the genome region manager. In certain embodiments, the set of CGH probes selected has a density specified by a user (e.g., a high density as compared to standard whole genome CGH probes). In certain embodiments, the subject systems contain a database that includes: genomic information (e.g., information employed by the genome region manager that allows it to identify a sub-genomic region of interest based on a sub-genomic region identifier provided by a user), at least one CGH probe group, and supporting information for the probes in the CGH probe group(s).

Aspects of the invention include methods of selecting a set of CGH probes specific for a sub-genomic region of interest. In certain embodiments, the subject method includes: providing a database containing genomic information, CGH probe group(s), and supporting information for each probe of the CGH probe group(s); identifying a sub-genomic region of interest based in part on the genomic information; and selecting a set of CGH probes specific for the sub-genomic region of interest based in part on the supporting information. In certain embodiments, the set of CGH probes selected has a specified density (e.g., a high density as compared to standard whole genome CGH probes). In certain embodiments, the set of selected CGH probes contains at least one probe from the CGH probe group in the database.

Also provided are methods for receiving a set of CGH probes specific for a sub-genomic region of interest employing the systems of the invention as well as computer program products for executing the subject methods.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

In the event that one or more of the incorporated literature and similar materials differs from or contradicts this application, including but not limited to defined terms, term usage, described techniques, or the like, this application controls.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As noted above, aspects of the invention include systems and methods for selecting a set of CGH probes specific for a sub-genomic region based on at least one sub-genomic region identifier, e.g., an identifier that has been input or selected by a user. Embodiments of the subject systems generally include the following components: (a) a communications module for facilitating information transfer between the system and one or more users, e.g., via a user computer, as described below; (b) a database comprising genomic information, at least one CGH probe group, and supporting information for each probe of the at least one CGH probe group; and (c) a processing module for performing one or more tasks involved in the CGH probe set selection methods of the invention.

In certain embodiments, the subject systems may be viewed as being the physical embodiment of a web portal, where the term “web portal” refers to a web site or service, e.g., as may be viewed in the form of a web page, that offers a broad array of resources and services to users via an electronic communication element, e.g., via the Internet.

In certain embodiments, the subject invention communicates to the user information relating to a selected set of CGH probes that are specific for a sub-genomic region of interest. For example, the subject invention can provide a display on a GUI that lists a set of probes selected using the systems and methods of the invention to a user for review (e.g., individually, as a group, or in any other convenient format). In certain embodiments a user is able to add, delete, alter or otherwise manipulate the set of CGH probes returned to suit their needs. In certain embodiments, the systems and methods provide a selected set of CGH probes as a synthesized product, e.g., a set of individual CGH probes or as the features (or a subset of the features) on an array that finds use in CGH assays as described herein. As such, the subject invention clearly provides useful information and reagents for performing CGH assays.

In certain embodiments, the subject systems are components of an array development system, including but not limited to those systems described in Published United States Application publication Nos. 20060116827; 20060116825 and 20060115822, as well as U.S. application Ser. Nos. 11/349,425; 11/349,398; 11/478,975; 11/479,014; 11/478,973; 11/494,980; 11/494,824; 11/495,042 and 11/495,331; the disclosures of which are herein incorporated by reference in their entirety.

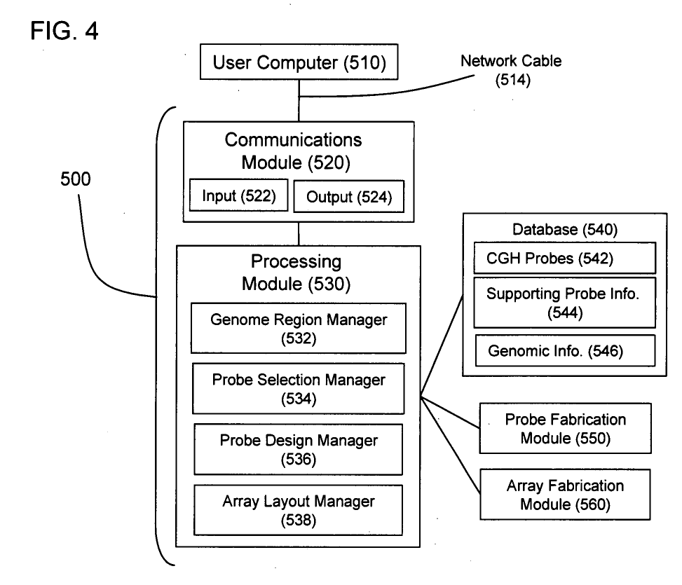

FIG. 4 provides a view of a probe selection system according to an embodiment of the subject invention. In FIG. 4, system 500 includes communications module 520 and processing module 530, where each module may be present on the same or different platforms, e.g., servers, as described above.

The communications module includes the input manager 522 and output manager 524 functional elements. Input manager 522 receives information from a user e.g., over the Internet. Input manager 522 processes and forwards this information to the processing module 530. These functions are implemented using any convenient method or technique. Another of the functional elements of communications module 520 is output manager 524. Output manager 524 provides information assembled by processing module 530 to a user, e.g., over the Internet. The presentation of data by the output manager may be implemented in accordance with any convenient methods or techniques. As some examples, data may include SQL, HTML or XML documents, email or other files, or data in other forms. The data may include Internet URL addresses so that a user may retrieve additional SQL, HTML, XML, or other documents or data from remote sources.

The communications module 520 may be operatively connected to a user computer 510, which provides a vehicle for a user to interact with the system 500. User computer 510, shown in FIG. 4, may be a computing device specially designed and configured to support and execute any of a multitude of different applications. Computer 510 also may be any of a variety of types of general-purpose computers such as a personal computer, network server, workstation, or other computer platform now or later developed. Computer 510 may include components such as a processor, an operating system, a graphical user interface (GUI) controller, a system memory, memory storage devices, and input-output controllers. There are many possible configurations of the components of computer 510 and some components are not listed above, such as cache memory, a data backup unit, and many other components.

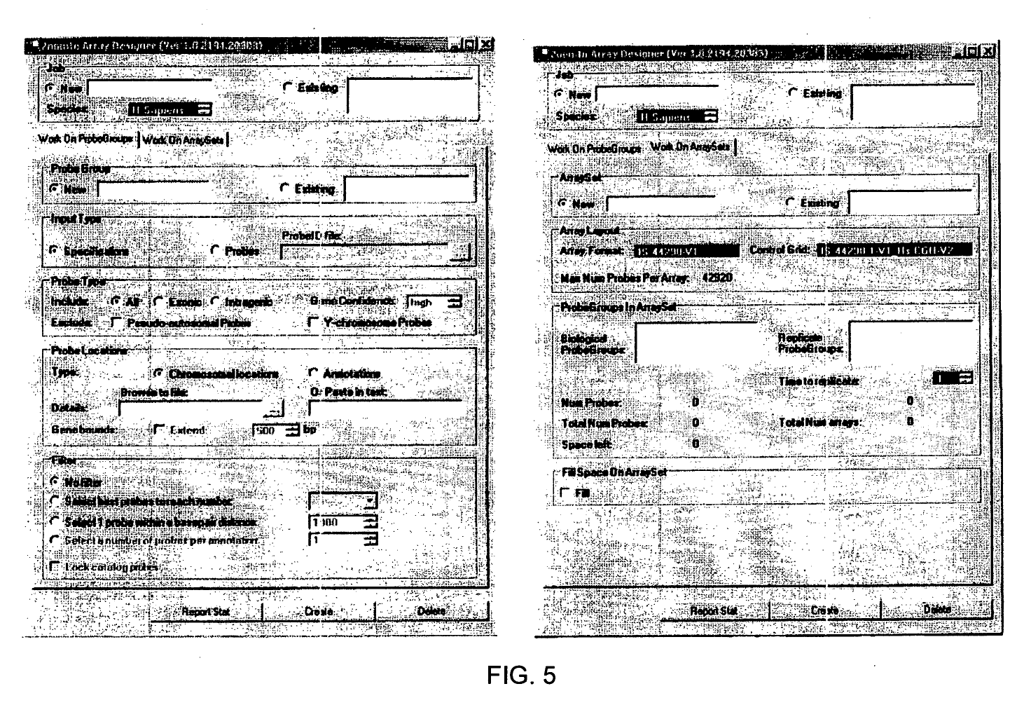

FIG. 5 shows exemplary screen shots of a GUI that finds use in the systems and methods of the subject invention. The left panel of FIG. 5 provides an exemplary GUI that can be employed by a user to enter input related to selecting a set of probes specific for a sub-genomic region of interest, whereas the right panel provides an exemplary GUI that can be employed by a user to provide input related to designing and/or fabricating a CGH array that includes a set of CGH probes selected according to the subject invention. As is evident from these exemplary GUI interfaces, a user may enter input in a variety of ways, including completing text fields, selecting from pull-down menus, uploading data files (e.g., CGH probe or array data files), highlighting (or checking) boxes, etc. These exemplary GUIs are not meant to limit in any way how input is entered or the types of fields/information employed (e.g., as input) in practicing the subject invention.

In certain embodiments, a computer program product is described comprising a computer usable/readable medium having control logic (computer software program, including program code) stored thereon. The control logic, when executed by the processor in the computer, causes the processor to perform functions described herein. In other embodiments, some functions are implemented primarily in hardware using, for example, a hardware state machine. Implementation of the hardware state machine so as to perform the functions described herein may be accomplished using any convenient method and techniques.

In certain embodiments, a user employs the user computer to enter information into and retrieve information from the system. As shown in FIG. 4, computer 510 is coupled via network cable 514 to the system 500. Additional computers of other users and/or administrators of the system in a local or wide-area network including an Intranet, the Internet, or any other network may also be coupled to system 500 via cable 514 (or other similar cables). It will be understood that cable 514 is merely representative of any type of network connectivity, which may involve cables, transmitters, relay stations, network servers, wireless communication devices, and many other components not shown, that are suitable for this purpose. Via user computer 510, a user may operate a web browser served by a user-side Internet client to communicate via Internet with system 500. System 500 may similarly be in communication over the Internet with other users, networks of users, and/or system administrators, as desired.

As reviewed above, the systems include various functional elements that carry out specific tasks on the platforms in response to information introduced into the system by one or more users. In FIG. 4, elements 532, 534, 536 and 538 represent four different functional elements of processing module 530. While four different functional elements are shown, it is noted that the number of functional elements may be more or less, depending on the particular embodiment of the invention. Representative functional elements that may be included in the processing module are now reviewed in greater detail below.

In certain embodiments, the subject system includes a genome region manager 532 as part of the processing module 530, which is configured to identify a sub-genomic region of interest in response to at least one sub-genomic region identifier input by a user. The genome region manager identifies the sub-genomic region based at least in part on genomic information 546 stored in the database 540. By “genomic information” is meant any type of information that can be used to identify a sub-genomic region of interest based on relevant input from a user. As such, genomic information includes, but is not limited to, one or more of: chromosomal information, polymorphism information, mutation information, transcriptome information, transcript mapping information, species information, and combinations thereof. By “chromosomal information” is meant any relevant chromosomal information, including but not limited to: chromosome number, chromosome map coordinates, chromosomal nucleic acid sequence, gene location, and the like. By “polymorphism” and “mutation information” is meant any information related to known differences in chromosome sequence or structure within a species that are associated with polymorphic regions of the chromosome or mutations that are related to specific characteristics or disease phenotypes (e.g., chromosomal translocations associated with a specific malignancy). By “transcriptome information” is meant any information on the nucleic acid sequence and structure of RNA transcripts in a cell, tissue or organism, and can include unique identifiers that correlate to specific transcripts (e.g., GenBank accession numbers, transcript name, etc.). By “transcript mapping information” is meant any information related to correlating an RNA transcript with its genomic origin, e.g., identifying intronic and exonic regions of a gene of interest.

As noted above, the genome region manager 532 is configured to identify a sub-genomic region of interest (i.e., a sub-genomic region for which a set of CGH probes is desired) based on one or more sub-genomic region identifiers input by a user. The selection may be done in view of a single sub-genomic region identifier or multiple sub-genomic region identifiers. As such, sub-genomic region identification in certain embodiments is carried out by the system in view of two or more sub-genomic region identifiers, such as three or more, four or more, five or more, 10 or more, etc. By “sub-genomic region identifier” is meant any identifier or information that can be employed in the system and methods of the invention to identify a sub-genomic region of interest. As such, sub-genomic region identifiers can include, but are not limited to: cytogenetic parameter, genomic sequence (e.g., all of part of a nucleic acid sequence for which a set of CGH probes are sought), gene identifier (e.g., GenBank accession number, common gene name, gene family, etc.) chromosomal location(s) (e.g., region, start and/or stop locations, etc.), transcript identifier (e.g., GenBank accession number, transcript name, etc.), species, chromosomal boundary (e.g., specific region to include or exclude from the boundaries of the sub-genomic region of interest, e.g., a flanking gene) and combinations thereof.

In certain embodiments, the sub-genomic region identifier employed by the probe selection manager includes information about the nucleic acid database to which the sub-genomic region identifier is drawn (or from which it was obtained). In other words, sub-genomic region identifiers of interest in certain embodiments include the identity of the originating database of the user input target sub-genomic region. The originating database of these embodiments may be a number of different types of databases, including but not limited to: nucleic acid database comprising the target nucleic acid is selected from one or more of: EST database, transcriptome database, genomic database, private database (e.g., databases maintained and administered by private entities), public database (e.g., Ensembl, RefSeq, Tiger HGI, NCBI EST, NCBI Unigene, and/or UCSC MRNA), curated database, and combinations thereof, etc. Such information may be used by the system to acquire information about the sub-genomic region of interest to include in the genomic information database. As such, in certain embodiments of the invention, the system retrieves genomic information relevant to the sub-genomic region of interest from one or more databases (as described above) based on the sub-genomic identifier input by a user.

In certain embodiments, the subject system includes a probe selection manager 532 as part of the processing module 530, which is configured to perform functions relating to selecting a set of CGH probes specific for a sub-genomic region of interest based in at least in part on supporting probe information 544 in database 540 (where the sub-genomic region of interest is identified as described above). By “set of CGH probes specific for a sub-genomic region of interest” or “set of CGH probes” is meant one or more CGH probes selected to predictably bind under certain hybridization conditions to a sub-genomic region of interest, meaning that the probe is selected to predictably bind or not bind to the sub-genomic region of interest in a CGH assay. As such, a set of CGH probes may contain one or multiple probes, including 2 or more probes, 4 or more probes, 10 or more probes, 30 or more probes, 50 or more probes, 100 or more probes, 500 or more probes and including up to 1000, 10,000 and 100,00 or more probes. As such, the number of probes in a set of CGH probes selected by employing the systems and methods of the invention can vary widely and is generally determined by a user. Indeed, because in certain embodiments the set of CGH probes are to be used in array-based CGH assays, the number of probes in a set can be very high (e.g., hundreds of thousands of probes or more).

By “supporting probe information” or “supporting information for a probe” and equivalents thereof is meant any information related to describing the characteristics of a probe, including, but not limited to: probe length, computational score (e.g., base composition, thermodynamic property, etc.), probe annotation (e.g., genome binding location, species, utility, e.g., for use in an assay for identifying a polymorphism, duplication or mutation, etc.), and combinations thereof.

In certain embodiments, the probe selection manager is configured to select probes from a database 540 that includes CGH probes 542. In certain of these embodiments, the CGH probes are provided in one or more CGH probe group(s), including, but not limited to: previously selected CGH probe groups, private CGH probe groups, public CGH probe groups, proprietary CGH probe groups, curated CGH probe groups, and combinations thereof. The probe selection manager may select any number of probes from any of the one or more CGH probe groups in selecting a set of CGH probes specific for a sub-genomic region of interest (as described above). As such, in the systems and methods of the invention, one probe, multiple probes, or all of the probes of one or more CGH probe groups (or any possible combination thereof) are selected to include in the set of CGH probes specific for the sub-genomic region of interest. In certain embodiments, a selected set of CGH probes is identical to a CGH probe group present in the CGH probe database 542. For example, the CGH probe database may include a previously-selected CGH probe group that meets the specifications input by a user. In this instance, the system may return this previously-selected probe group to the user.

In certain embodiments, processing module 530 includes a probe design manager 536 configured to design at least one probe specific for the sub-genomic region of interest. In certain of these embodiments, the probe selection manager 534 is configured to include at least one of the probes designed by the probe design manager 536 in the set of CGH probes specific for the sub-genomic region of interest returned to the user. In certain embodiments, the system is configured to allow the user to indicate to the system (e.g., by checking a box on a graphic displayed on a GUI) to employ the probe design manager 536 in selecting the set of CGH probes, whereas in certain other embodiments, the system is configured to automatically employ the probe design manager 536.

In certain embodiments, the set of CGH probes is selected based in part on at least one probe-specific parameter input by a user. By “based in part on” is meant that the CGH probe selection protocol employed by the system uses the one or more input probe-specific parameters as a guiding basis for selecting a set of CGH probes, e.g., in choosing probes from a database and/or designing probes. The probe selection manager may select a set of CGH probes (e.g., by choosing from existing probes and/or designing probes) for a sub-genomic region based on any of a variety of different probe-specific parameters. The selection may be done in view of a single probe-specific parameter or multiple probe-specific parameters. As such, CGH probe set selection in certain embodiments is carried out by the system in view of two or more probe-specific parameters, such as three or more, four or more, five or more, 10 or more, etc. Probe-specific parameters of interest include, but are not limited to: density of probes, types of probes, probe boundary, probe interval, minimum number of probes, maximum number of probes, probe computational score, gene confidence level, and combinations thereof.

By “density of probes” is meant the number of CGH probes per length of genomic sequence (e.g., probes/megabase (Mb), where Mb=1×10⁶ contiguous base pairs of a double-stranded nucleic acid, e.g., genomic DNA). In certain embodiments, the probe density indicated by a user is a high density (e.g., a density that is higher for the sub-genomic region of interest than for the genome as a whole). As such, in certain embodiments, the probe density ranges from 1 probe/Mb to 10,000 probes/Mb, including 10 probes/Mb or more, 50 probes/Mb or more, 250 probes/Mb or more, 1,000 probes/Mb, 5,000 probes/Mb, or more. In certain embodiments, the probe density may be consistent throughout the sub-genomic region of interest or, alternatively, may vary within the sub-genomic region of interest. In general, the overall and local probe density of a set of CGH probes selected by the system will depend on the totality of the parameters specified by the user and/or considered by the system during the selection process, as discussed in further detail below.

By “types of probes” is meant any parameter that describes to what type of region a probe binds (e.g., intron specific, exon specific, intergenic, intragenic, etc.). By “probe interval” is meant the minimum and/or maximum nucleotide distance between adjacent probes of a set. By “probe boundary” is meant the maximum nucleotide distance outside of the sub-genomic region of interest that the outermost probes of the set can be located. By “probe number” is meant an exact or approximate total probe number of probes in a set. By “computational score” is meant any calculable factor specific for a probe (e.g., base composition and/or thermodynamic property, and combinations thereof. Base composition parameters of interest include, but are not limited to: percent A, percent T, percent G, percent C, percent GC, percent AmC, percent TmG, number of poly X (where X is any nucleotide) and number of poly 5′ A. Suitable ranges for each of these parameters may vary. In certain embodiments, percent A is chosen to range from 10% to 60%, percent T is chosen to range from 10% to 60%, percent G is chosen to range from 10% to 60%, percent C is chosen to range from 10% to 60%, percent GC is chosen to range from 30% to 70%, percent AmC is chosen to range from 0% to 20%, percent TmG is chosen to range from 0% to 20%, number of poly X is chosen to range from 2 to 8 and number of poly 5′A is chosen to range from 2 to 8. By “thermodynamic property” is meant any thermodynamic property that pertains to the tightness or strength of binding between a probe and a target. Non-limiting examples of such thermodynamic properties include ΔG, melting temperature (Tₘ), and ΔH. The thermodynamic property may be calculated using any convenient method. In certain embodiments, the thermodynamic property is calculated by assuming specific probe/candidate target binding conditions. For example, calculating a thermodynamic property of binding between a nucleic acid probe and a nucleic acid target can be done by assuming that the binding is done under stringent hybridization conditions (such hybridization conditions are described in detail, above).

As can be seen form the above description, certain of the probe-specific parameters may impact one another in guiding a system of the invention in selecting a set of CGH probes specific for a sub-genomic region of interest. For example, providing a desired density of probes and a minimum probe interval may return a distinct set of probes as compared to only specifying one of probe density or minimum probe interval. As such, certain parameters may need to be adjusted in subsequent CGH probe set queries to obtain an “optimal” set of CGH probes specific for a sub-genomic region of interest (where by “optimal” is merely meant that a user of the system and/or method of the invention has deemed the subject set of CGH probes “optimal”).

In certain embodiments, the probe-selection manager 534 is configured to select a set of CGH probes based in part on at least one experimental parameter input by a user. Experimental parameters include, but are not limited to: target sample preparation, assay format, assay parameter, and combinations thereof. For example, experimental parameters may include one or more of the following: labeling reaction (e.g., direct labeling reaction, linear amplification labeling reaction, and PCR-based labeling reaction, etc.), type of label (e.g., fluorescent label, radioactive label, FRET label, and enzymatic label, etc.), hybridization conditions (e.g., buffer composition, buffer pH, temperature of hybridization, duration of hybridization, concentration of probe and/or target, etc.). As such, in these embodiments, the system and methods of the invention include selecting a set of CGH probes in view of one or more input experimental parameters, e.g., as described above, using any convenient protocol or algorithm. For example, the probe selection manager 534 may be configured to employ a set of decision rules which determine selection criteria based on input experimental parameters. The decision rules may be developed using any convenient criteria, such as empirically determined functional characteristics of a probe previously employed in a CGH assay, e.g., data on how a given probe has performed under a given set of CGH hybridization assay conditions, data on how targets for a CGH probe have been generated, etc. As such, in certain embodiments, the probe selection manager makes “informed” probe selection decisions based on the experimental parameters input by a user. For example, a user may input a target cenomic sequence and specify that it is to be employed in an array-based CGH assay under a given set of hybridization conditions. (The experimental parameters may be input using any convenient format, such as a GUI, e.g., where the user may select from a pull down menu and/or input parameters manually). The probe selection manager may then choose a set of CGH probes for the sub-genomic region of interest based on its predetermined ability to function well in an array-based CGH assay under the hybridization conditions specified by the user.

As summarized above, the probe selection manager may select a probe by choosing from a database of candidate probes and/or designing a probe. Accordingly, systems in accordance with the invention may include a probe database 540 as either part of the system or in communication with the system. In these embodiments, the probe selection manager 534 is configured to retrieve one or more probe sequences from among probe sequences stored in the CGH probe database 542, e.g., automatically or when prompted by the user. Systems in accordance with the invention may also include, either in addition to or instead of the CGH probe database, a probe design manager 536, which designs one or more CGH probes for the probe selection manager, where the CGH probes are designed based on the input experimental parameter(s). As such, systems in accordance with the invention may include a probe design manager 536 configured to design one or more CGH probe sequences, e.g., automatically or when prompted by the user.

The probe design manager 536 may employ any convenient probe design algorithm(s) to design CGH probe(s) to include in the set of CGH probes specific for the sub-genomic region of interest. Probe design algorithms of interest include, but are not limited to: those described in U.S. Pat. Nos. 6,251,588 and 6,461,816, as well as published US Application No. 20060110744; the disclosures of which probe design algorithms are incorporated herein by reference. In certain embodiments, the probe design manager 536 operates the design algorithm using default settings for various design parameters. In yet other embodiments, the probe design manager 536 operates the design algorithm using one or more parameters that have been set by a user, e.g., through use of an appropriate graphical user interface, such that the probe design manager 536 designs the one or more CGH probes for the set of CGH probes based in part on one or more parameter provided by the user.

In certain embodiments, the system includes a user domain, wherein any of the sets CGH probes selected by the probe selection manager are stored, e.g., automatically or when prompted by a user.

In certain embodiments, the system includes a probe fabrication module 550, e.g., that fabricates a probe based on one or more sets of CGH probe sequences selected by the probe selection manager (e.g., stored in the user domain), where fabrication may occur automatically or when prompted by the user.

In certain embodiments, the system includes an array fabrication module 560, e.g., that fabricates an array that includes one or more sets of CGH probes specific for a sub-genomic region of interest (or other probes) based on CGH probe sequences selected by the probe selection manager (e.g., stored in the user domain), where fabrication may occur automatically or when prompted by a user. Exemplary array fabrication systems and methods are described in U.S. Pat. Nos. 6,613,893; 6,599,693; 6,587,579; 6,420,180; and 6,180,351 incorporated herein by reference in their entirety.

In certain embodiments the system includes an array layout manager 538 configured to generate array layouts, where a set of CGH probes selected by the systems are employed. In certain embodiments, the array layouts generated by the array layout manager may be returned to the user via in electronic format (e.g., for inspection and/or alteration) and/or may be employed as a template for fabricating the array using the array fabrication module 560. In certain embodiments, the array layout manager 538 is configured to include one or more of the following features (e.g., probes) in an array layout: replicate probes, normalization control probes, negative control probes, positive control probes, CGH probes specific for regions outside of said sub-genomic region of interest, and combinations thereof (see, e.g., PCT application serial no. US07/02127, which describes normalization control probe sets). The inclusion of such additional probes may accomplished by any convenient method, including being specified by a user, added automatically by the array layout manager, or the like.

In certain embodiments, the array layout manager has similar functionality to the array layout manger described in copending application Ser. No. 11/001,700, having U.S. Patent Application Publication No. 2006/0116825, which is incorporated herein by reference in its entirety. In certain of these embodiments, the array layout manager 538 comprises an array layout developer, where the array layout developer includes a memory having a plurality of rules relating to array layout design and is configured to develop an array layout based on the application of one or more of the rules to information that includes array request information received from a user.

CGH applications of interest include hybridization assays in which the nucleic acid arrays of the subject invention are employed. In these assays, a sample of target nucleic acids is first prepared (e.g., from the genomic samples of interest), where preparation may include labeling of the target nucleic acids with a label, e.g., a member of a signal producing system. Following sample preparation, the sample is contacted with the array under hybridization conditions, whereby complexes are formed between target nucleic acids that are complementary to CGH probe sequences attached to the array surface. The presence of hybridized complexes is then detected. Exemplary methods of using arrays in CGH applications are described in U.S. Patent Application Publication Nos. 2005/0233338 (having application Ser. No. 10/828,892), 2004/0191813 (having application Ser. No. 10/744,595), and 2004/0241658 (having application Ser. No. 10/448,298), each of which is incorporated herein by reference.

As such, in using an array having a set of CGH probes selected by the system and method of the present invention, the array will typically be exposed to a sample (for example, fluorescently labeled samples) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934. However, arrays may be read by any other method or apparatus than the foregoing, with other reading methods including other optical techniques (for example, detecting chemi-luminescent or electro-luminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the output manager further provides a user with information regarding how to purchase the selected set of CGH probe sequences, e.g., alone or in an array. In certain embodiments, the information is provided in the form of an email. In certain embodiments, the information is provided in the form of web page content on a graphical user interface in communication with the output manager. In certain embodiments, the web page content provides a user with an option to select for purchase one or more synthesized CGH probe sets. In certain embodiments, the web page content includes fields for inputting customer information. In certain embodiments, the system can store the customer information in the memory. In certain embodiments, the customer information includes one or more purchase order numbers. In certain embodiments, the customer information includes one or more purchase order numbers and the system prompts a user to select a purchase order number prior to purchasing the one or more synthesized probe sequences.

In certain embodiments, in response to the purchasing, one or more CGH probe set sequences are synthesized on an array. In certain embodiments, the methods include ordering synthesized probe(s) that include the sequences of the selected set of CGH probes. In certain embodiments, the synthesized set of CGH probes are synthesized on an array. In certain embodiments, the inputting is via a graphical user interface in communication with the system.

In certain embodiments, the user may choose to obtain an array having the selected CGH probe set present therein. As such, the CGH probe set can be included in an array layout, and an array fabricated according to the array layout that includes the CGH probe set. In certain embodiments, the user may specify the location of the CGH probe set in the product layout (as well as other features/probes not part of the CGH probe set, e.g., control probes, normalization probes, etc.). Specifying may include choosing a particular location in a given layout, or choosing from a section of system-provided array layout options in which the probe is present at various locations. Array fabrication according to an array layout can be accomplished in a number of different ways. With respect to nucleic acid arrays in which the immobilized nucleic acids are covalently attached to the substrate surface, such arrays may be synthesized via in situ synthesis in which the nucleic acid ligand is grown on the surface of the substrate in a step-wise fashion and via deposition of the full ligand, e.g., in which a presynthesized nucleic acid/polypeptide, cDNA fragment, etc., onto the surface of the array.

Where the in situ synthesis approach is employed, conventional phosphoramidite synthesis protocols are typically used. In phosphoramidite synthesis protocols, the 3′-hydroxyl group of an initial 5′-protected nucleoside is first covalently attached to the polymer support, e.g., a planar substrate surface. Synthesis of the nucleic acid then proceeds by deprotection of the 5′-hydroxyl group of the attached nucleoside, followed by coupling of an incoming nucleoside-3′-phosphoramidite to the deprotected 5′ hydroxyl group (5′-OH). The resulting phosphite triester is finally oxidized to a phosphotriester to complete the internucleotide bond. The steps of deprotection, coupling and oxidation are repeated until a nucleic acid of the desired length and sequence is obtained. Optionally, a capping reaction may be used after the coupling and/or after the oxidation to inactivate the growing DNA chains that failed in the previous coupling step, thereby avoiding the synthesis of inaccurate sequences.

In the synthesis of nucleic acids on the surface of a substrate, reactive deoxynucleoside phosphoramidites are successively applied, in molecular amounts exceeding the molecular amounts of target hydroxyl groups of the substrate or growing oligonucleotide polymers, to specific cells of the high-density array, where they chemically bond to the target hydroxyl groups. Then, unreacted deoxynucleoside phosphoramidites from multiple cells of the high-density array are washed away, oxidation of the phosphite bonds joining the newly added deoxynucleosides to the growing oligonucleotide polymers to form phosphate bonds is carried out, and unreacted hydroxyl groups of the substrate or growing oligonucleotide polymers are chemically capped to prevent them from reacting with subsequently applied deoxynucleoside phosphoramidites. Optionally, the capping reaction may be done prior to oxidation.

With respect to actual array fabrication, in certain embodiments, the user himself may produce an array having the generated array layout. In yet other embodiments, the user may forward the array layout to a specialized array fabricator or vendor, which vendor will then fabricate the array according to the array layout.

In yet other embodiments, the system may be in communication with an array fabrication station, e.g., where the system operator is also an array vendor, such that the user may order an array directly through the system. In response to receiving an order from the user, the system will forward the array layout to a fabrication station, and the fabrication station will fabricate the array according to the forwarded array layout.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, light directed fabrication methods may be used, as are known in the art. Interfeature areas need not be present particularly when the arrays are made by light directed synthesis protocols.

The invention also provides programming, e.g., in the form of computer program products, for use in practicing the CGH probe set selection methods of the invention. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. Any convenient medium or storage method can be used to create a manufacture that includes a recording of the present programming/algorithms for carrying out the above described methodology.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A system for selecting a set of comparative genome hybridization (CGH) probes specific for a sub-genomic region, said system comprising:
(a) a communication module comprising an input manager for receiving input from a user and an output manager for communicating output to a user;
(b) a database comprising:
(i) genomic information;
(ii) at least one CGH probe group; and
(iii) supporting information for each probe of said at least one CGH probe group; and
(c) a processing module comprising:
(i) a genome region manager configured to identify a sub-genomic region of interest in response to at least one sub-genomic region identifier input by said user, wherein said genome region manager identifies said sub-genomic region based in part on said genomic information; and
(ii) a probe selection manager configured to select a set of CGH probes specific for said sub-genomic region;
wherein said probe selection manager selects said set of CGH probes based in part on said supporting information and said set of CGH probes comprises at least one probe from said at least one CGH probe group.

2. The system of claim 1, wherein said probe selection manager is further configured to select said set of CGH probes based on at least one probe-specific parameter input by said user.

3. The system of claim 2, wherein said at least one probe-specific parameter comprises one or more of: density of probes, type of probes, probe boundary, probe interval, minimum number of probes, maximum number of probes, probe computational score, gene confidence level, and combinations thereof.

4. The system of claim 3, wherein said density of probes ranges from 1 probe/Mb to 10,000 probes/Mb.

5. The system of claim 3, wherein said type of probes is selected from one or more of: intron specific, exon specific, intergenic, and intragenic.

6. The system of claim 1, wherein said at least one CGH probe group comprises one or more of: previously selected CGH probe group, private CGH probe group, public CGH probe group, proprietary CGH probe group, and curated CGH probe group.

7. The system of claim 1, wherein said supporting information comprises one or more of: probe length, computational score, and probe annotation.

8. The system of claim 1, wherein said genomic information comprises one or more of: chromosomal information, polymorphism information, mutation information, transcriptome information, transcript mapping information, and species information.

9. The system of claim 1, wherein said sub-genomic region identifier comprises one or more of: cytogenetic parameter, genomic sequence, gene identifier, chromosomal location, transcript identifier, species, and chromosomal boundary.

10. The system of claim 1, wherein said probe selection manager is further configured to select said set of CGH probes based in part on at least one experimental parameter input by said user.

11. The system of claim 10, wherein said experimental parameter comprises one or more of: target sample preparation, assay format, assay parameter, and combinations thereof.

12. The system of claim 1, wherein said processing module further comprises a probe design manager, wherein said probe design manager is configured to design at least one probe to include in said set of CGH probes when prompted by said user.

13. The system of claim 1, wherein said system further comprises a user domain configured to store said set of CGH probes when prompted by said user.

14. The system of claim 1, wherein said system further comprises a probe fabrication module configured to fabricate said set of CGH probes when prompted by said user.

15. The system of claim 1, wherein said processing module further comprise an array layout manager configured to design an array layout comprising said set of CGH probes when prompted by said user.

16. The system of claim 15, wherein said array layout manager is further configured to include in said array layout one or more of: replicate probes, normalization control probes, negative control probes, positive control probes, CGH probes specific for regions outside of said sub-genomic region of interest, and combinations thereof.

17. The system of claim 15, wherein said system further comprises an array fabrication module configured to fabricate an array based on said array layout.

18. The system of claim 1, wherein said system further comprises a graphical user interface (GUI) linked to said communication module, wherein said GUI is configured to prompt said user for input and to display output of said system to said user.

19. A method of receiving a set of CGH probes specific for a sub-genomic region, said method comprising:
(a) inputting an identifier for a sub-genomic region into the system of claim 1; and
(b) receiving a set of CGH probes specific for said sub-genomic region.

20. A method of selecting a set of CGH probes specific for a sub-genomic region, said method comprising:
(a) providing a database comprising:
(i) genomic information;
(ii) at least one CGH probe group; and
(iii) supporting information for each probe of said at least one CGH probe group;
(b) identifying a sub-genomic region based in part on said genomic information;
(c) selecting a set of CGH probes specific for said sub-genomic region based in part on said supporting information, wherein said set of CGH probes comprises at least one probe from said CGH probe group.

21. The method of claim 20, wherein said identifying step comprises:
(i) providing at least one sub-genomic region identifier, wherein said sub-genomic region identifier comprises one of more of: cytogenetic parameter, genomic sequence, gene identifier, chromosomal location, transcript identifier, organism, chromosomal boundary, and combinations thereof; and
(ii) identifying said sub-genomic region based in part on said at least one sub-genomic region identifier.

22. The method of claim 20, wherein said selecting step further comprises:
(i) specifying at least one probe-specific parameter; and
(ii) selecting said set of CGH probes based in part on said probe specific parameter.

23. The method of claim 20, wherein said obtaining step further comprises designing one or more probe in said set of CGH probes using a probe design algorithm.

24. The method of claim 20, wherein said selecting step further comprises submitting said set of CGH probes to a pairwise reduction algorithm.

25. The method of claim 20, wherein said selecting step is further based on at least one experimental parameter.

26. The method of claim 25, wherein said experimental parameter comprises one or more of: target sample preparation, assay format, assay parameter, and combinations thereof.

27. The method of claim 20, wherein said method further comprises storing said set of CGH probes in said database as one of said at least one CGH probe group.

28. The method of claim 21, wherein said sub-genomic region identifier is provided using a graphical user interface (GUI).

29. The method of claim 22, wherein said probe specific parameter is specified using a GUI.

30. The method of claim 20, wherein said set of CGH probes is displayed on a GUI.

31. A method of fabricating an array, said method comprising:
a) selecting a set of CGH probes specific for a sub-genomic region according to the method of claim 20;
b) designing an array layout comprising said set of CGH probes; and
c) fabricating an array based on said array layout.

32. A computer program product comprising a computer readable storage medium having a computer program stored thereon, wherein said computer program, when loaded onto a computer, operates said computer to select a set of CGH probes specific for a sub-genomic region of interest identified by a user based in part on one or more sub-genomic region identifier specified by said user.
